(19) United States
(12) Patent Application Publication
(10) Pub. No.: US 2005/0234004 A1
(43) Pub. Date: Oct. 20, 2005

(54) Compositions and methods of use of W-peptides

(76) Inventors: Brett Premack, San Francisco, CA (US); Thomas Schall, Palo Alto, CA (US)

Correspondence Address: BRINKS HOFER GILSON & LIONE, P.O. BOX 10395, CHICAGO, IL, 60610, US

(21) Appl. No.: 11/043,020

(22) Filed: Jan. 25, 2005

Related U.S. Application Data
(60) Provisional application No. 60/539,665, filed on Jan. 26, 2004.

(51) Int. Cl. A61K 48/00

(52) U.S. Cl. 514/44

(57) ABSTRACT

The present invention relates to compositions and methods to modulating immune responses, such as those elicited by vaccination with W peptides. The compositions and methods are useful for, among other things, vaccine formulation for therapeutic and prophylactic vaccination (immunization) and for production of useful antibodies (e.g., monoclonal antibodies, for therapeutic or diagnostic use).

### RELATED APPLICATIONS

The present patent document claims the benefit of the filing date under 35 U.S.C. §119(e) of Provisional U.S. Patent Application Ser. No. 60/539,665, filed Jan. 26, 2004, which is hereby incorporated by reference.

### FIELD OF THE INVENTION

The invention relates to compositions and methods relating to modulating immune responses, such as those elicited by vaccination. The compositions and methods are useful for, among other things, vaccine formulation for therapeutic and prophylactic vaccination (immunization) and for production of useful antibodies (e.g., monoclonal antibodies, for therapeutic or diagnostic use).

### BACKGROUND

To date, many vaccines have been developed to prevent infection from a wide variety of agents, such as infectious microorganisms (bacteria and viruses), toxins, and even tumors. However, despite significant advances, many infectious agents are still free to prey on susceptible individuals because no effective vaccines exist.

Vaccination exploits the immune system, which comprises leukocytes (white blood cells (WBCs): T and B lymphocytes, monocytes, eosinophils, basophils, and neutrophils), lymphoid tissues and lymphoid vessels. Key players in the adaptive immune response to foreign invaders are also the antigen presenting cells (APCs), such as macrophages, activated B cells and dendritic cells. Dendritic cells are especially important in the immune response. Immature or resting dendritic cells reside in epithelial layers, phagocytosing foreign material (called antigens). These dendritic cells become activated by tumor necrosis factor (TNF) secreted by nearby macrophages that have been stimulated by the foreign material. These activated dendritic cells, laden with foreign antigens, travel through the lymphatic system to the nearest lymph node. There, resting naïve (unexposed to antigen) T cells whose antigen-specific receptors recognize the foreign antigen are activated, and the immune system is triggered into action.

The concept of vaccination is to generate the same types of host-protective immune responses without exposing the individual to the pathology-inducing foreign agent (such as a pathogen or tumor). Such immune responses may be, for example, cell-mediated and/or antibody based.

Leukocyte recruitment at the site of inflammation and infection is dependent on the presence of a gradient of chemotactic factors or chemoattractants. Various chemoattractants, including several chemokines, are able to activate and recruit phagocytic cells to the site of infection. Many chemoattractants stimulate leukocytes via the activation of a seven-transmembrane, G-protein coupled receptors (GPCRs), including a pertussis toxin (PTX)-sensitive G protein coupled receptor (Bokoch, 1995). Upon binding to its corresponding cell surface receptor, a chemoattractant induces intracellular calcium mobilization, cytoskeletal rearrangement, exocytosis, histamine release, receptor induction, adhesion, the production of bioactive lipids and the activation of the respiratory burst system via NADPH oxidase activation (Bokoch, 1995; Prieschl EE et al., 1995; and Baggiolini M. et al., 1994).

While vaccination can be accomplished with attenuated or dead infectious agents, the safest vaccinations are those that provoke an immune response to a subset of isolated antigens or epitopes, expressed by the foreign agent. However, many such antigens are by themselves weakly immunogenic or incompetent for instigating a strong immune response. To modulate the effectiveness of such antigens, adjuvants are often added to vaccine compositions. Examples of adjuvants include oil emulsions of dead mycobacteria (Freund's complete), other dead bacteria (e.g., B. pertussis), bacterial polysaccharides, bacterial heat-shock proteins or bacterial DNA. While effective, many of these adjuvants cause significant inflammation and are-not suitable for human administration.

Present immunization methods are not effective for all antigens, for all individuals, or for eliciting all forms of protective immunity. In addition, the number of useful adjuvants is small and directed mainly to antibody-related immunity and not to cell-mediated immunity. Moreover, there is a considerable lag time from immunization until the immune system provides protection for the subject. Improved vaccine compositions and/or effective safe adjuvants capable of modulating cell-mediated responses as well as antibody, would greatly aid current vaccination efforts.

Chemoattractants, activate leukocytes via PTX-sensitive G protein(s) by binding to the specific cell-surface receptors belonging to the family of G protein-coupled seven-transmembrane “chemoattractant receptors.” On binding their cognate ligands, chemoattractant receptors transducer an intracellular signal through the associates trimeric G protein, resulting in a rapid increase in intracellular calcium concentration and a downstream response.

Chemoattractants include the so called “W Peptides or W-tide(s).” These W-tides are high affinity ligands of Formyl Peptide Receptor-Like 1(FPRL1). W-tides act as ligands to a chemoattractant receptor, such as FPRL1, causing intracellular calcium flux in leukocytes and inducing chemotactic migration of human monocytes. The concept of chemotaxis (otherwise known as cell migration) is clear in the art. In addition to monocytes, W-tides effectively attract other types of leukocytes, namely neutrophils.

To date, at least twenty eight W-tides have been identified and described. Examples of W-tides and protein and peptides comprising W-tide sequences that may be used with the present invention include, but are not limited to, the following W-tides which are incorporated by reference herein. W-tides such as HFYLPM (SEQ ID NO: 1) and MFYLPM (SEQ ID NO: 2) were identified by Bae et al. (Bae et al., 2001). Additionally, the synthetic hexapeptides HFYLPm (SEQ ID NO: 3) and WKYMVm (SEQ ID NO: 4) are examples of W-peptides that may be used with the present invention (Baek SH et al., 1996). A recent publication, WO 03/064447 A2, further identifies twenty four variants of WKYMVm polypeptide, SEQ ID NOS: 5-28, which may be used with the present invention.

A search of the SWISS-PROT and TrEMBL databases did not identify proteins carrying the same exact sequences as W-tides HFYLPM (SEQ ID NO: 1) and MFYLPM (SEQ ID NO: 2), however it was found that several viral proteins such as the major capsid protein of the pseudorabies virus contain the X(F/K)Y(L/M)(V/P)M sequence. However, the significance of this sequence homology is still unclear (Bae et al., 2001).

FPRL1 is of the N-formyl peptide receptor (FPR) family of receptors referred to as “FPR class” or “FPR members”, which also includes FPRL2 (Le et al., 2001). The FPR class are G-protein-coupled receptors which have seven transmembrane domains. FPR members are typically found on human phagocytic cells but they have also been identified on hepatocytes, and cytokine stimulated epithelial cells. Many other cell types may have FPR members.

FPR and FPRL1 receptors interact with a number of ligands, as illustrated in Table 1 below by Le et al., 2001.

TABLE 1
Agonists and antagonists of formyl peptide receptors.

| LIGANDS | FPR | FPRL1 |
|---|---|---|
| Agonists | | |
| Bacterial peptide | | |
| fMLF | ++++ | + |
| HIV-1 Env domains: | | |
| T20/DP176 | ++++ | + |
| T21/DP107 | +++ | ++++ |
| N36 | − | +++ |
| F peptide | − | +++ |
| V3 peptide | − | +++ |
| Host-derived agonists: | | |
| LL-37 | − | ++++ |
| SAA | − | ++++ |
| Aβ₄₂ | ++ | ++++ |
| PrP106-126 | − | ++++ |
| Annexin I | +++ | − |
| Mitochondrial peptide | − | ++++ |
| LXA4 | − | ++++ |
| Peptide library derived agonists | | |
| W peptide | +++ | ++++ |
| MMK-1 | − | ++++ |
| Antagonists: | | |
| Boc-FLFLF | ++ | ? |
| CsH | +++ | − |
| Deoxycholoc acid (DCA) | +++ | +++ |
| Chenodeoxycholic acid (CDCA) | +++ | +++ |

FPR and FPRL1 are expressed by monocytes and neutrophils and are clustered on human chromosome 19q13 (Bao et al., 1992; and Durstin et al., 1994). FPRL1 was identified and molecularly cloned from human phagocytic cells by low stringency hybridization of the cDNA library with the FPR sequence and was initially defined as an orphan receptor (Gao and M. Murphy, 1993; Murphy et al., 1992; Ye et al., 1992; and Nomura et al., 1993). Another homolog of FPR receptor, FPRL2 was also described (Bao et al., 1992); however, no ligands have been identified for this receptor (Le, et al., 2001). FPRL1 possesses 69% identity at the amino acid level to FPR (Prossnitz and Ye, 1997; and Murphy et al., 1996). Many more FPR members, including FPRL2, may be present and can be rapidly identified by using the cloning methods detailed in the references cited above and the functional assays known in the art.

Based on their ability to recognize chemotactic peptides, FPR, FPRL2 and FPRL1 have been proposed to play an important role in host defense against microbial invasion. In fact, stimulation of phagocytic leukocyte by a bacterial ligand of FPR receptor, fMLF, can elicit shape change, chemotaxis, adhesion, phagocytosis, release of superoxide anions, and granule contents. In addition, fMLF has been shown to stimulate the activation of NFκB (Drowning D D, et al., 1997), and production of inflammatory cytokines by phagocytes (Murphy P M, 1996) and astrocytoma cell lines (Le Y. et al, 2000). Furthermore, peptide library-derived agonists, such as W-tides, were found to modulate and/or enhance the immune responses in vitro (Bae et al., 2001). Mobilization of phagocytes and increased production of bactericidal mediators are necessary for a rapid host response to invading pathogenic microorganism.

FPR and FPRL1 have also been considered as players in several devastating diseases, including the HIV-1 infection (Le et al., 2001) and systemic lupus erythematosus (SLE).

Furthermore, recent findings that FPRL1 is a functional receptor for at least three forms of amyloidogenic protein and peptide agonists, SAA, Aβ₄₂, and PrP106-126, indicate that FPRL1 may play a significant role in several disease states, including Alzheimer's disease (AD) and prion disease such as Creutzfeldt-Jakob disease (CJD). Although the causes of AD and prion disease are unknown, the identification of FPRL1 as a functional receptor for Aβ₄₂, and the prion protein fragment PrP106-126 nevertheless provides a molecular link in the chain of proinfammatory responses observed in AD and prion diseases. For example, the activation of FPRL1 may help direct the migration and accumulation of mononuclear phagocytes to sites containing elevated levels of these chemotactic agonists. The infiltrating phagocytes may ingest amyloidogenic proteins and fragments through internalization of the ligand-FPRL1 complex.

Based on the discovery that W-peptides act as effective FPRL1 receptor ligands that can modulate the immune response, the present invention is directed to providing compositions and methods for modulating an immune response using at least one W-peptide and at least one antigen.

### SUMMARY

In one aspect, the invention provides a method of modulating an immune response in a subject including administering at least one W-peptide or a conservative variant or a functional fragment thereof and at least one antigen in an amount sufficient to modulate an immune response in a subject.

In another aspect, the invention provides a method of producing antibodies to an antigen in a subject including administering to the subject at least one antigen and at least one W-peptide or a conservative variant or a functional fragment thereof, in an amount sufficient to elicit production of antibodies to the antigen in the subject.

In yet another aspect, this invention provides a composition including at least one W-peptide or conservative variant or a functional fragment thereof, at least one antigen, and a pharmaceutically acceptable carrier.

### DETAILED DESCRIPTION OF THE PRESENTLY PREFERRED EMBODIMENTS

It is to be understood that this invention is not limited to the particular methodology, protocols, cell lines, animal species or genera, constructs, or reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

The invention provides methods for modulating an immune response in a subject that includes administering to the subject at least one antigen and at least one W-peptide in an amount sufficient to modulate the immune response in the subject.

The methods of the invention, when modulating an immune response, include administering W-tide compositions containing the antigens of interest. In some cases, the antigen-containing composition is administered first, followed by administration of a W-tide-containing composition. In other embodiments the antigen-containing composition is administered last. The different compositions may be administered simultaneously, closely in sequence, or separated in time, e.g., one hour to two weeks or more.

The invention also provides compositions that include at least one W-tide or a conservative variant or a functional fragment thereof, at least one antigen, and a pharmaceutically acceptable carrier.

New methods and compositions are now provided for therapeutic and prophylactic immunization (i.e., the deliberate provocation, enhancement, intensification or modulation of an adaptive and/or innate immune response). Particular advantages include one or more of the following:

(1) an accelerated immune response following administration of the W-tide and the antigen, as compared to sole administration of the antigen;

(2) greater sensitivity to small amounts of antigen (e.g., toxin or pathogen) or antigens that do not habitually provoke strong immune responses, and

(3) more effective anti-tumor therapies.

While current vaccines are effective against many pathogenic agents, some dangerous pathogens (such as HIV, cancer and tumor cells, etc.) as of yet do not have suitable vaccines. In some instances, the difficulties partly stem from the properties of candidate foreign antigens, such as insolubility of HIV glycoproteins (e.g., gp120) or the poor immunogenicity of tumor antigens. A composition that improves the recognition of these antigens as foreign and modulates immune responses will be helpful to prepare new and effective vaccines.

The inventors have discovered that W-tides are able to modulate the immune responses in vitro and in vivo. Without intending to be bound by a particular mechanism, it is believed that the W-tide polypeptides promote an immune reaction to the antigen by binding to the FPRL1 receptor and influencing cellular responses, including but not limited to, signal transduction, leukocyte migration, immune system response, inflammatory responses, infection, organ rejection, arthritis, atherosclerosis, and neoplasia.

Definitions:

The following definitions are provided in order to aid the reader in understanding the detailed description of the present invention.

“Modulating an immune response” means affecting the classes and subtypes of produced immunoglobulins (Ig's) or cytokines, and/or the number and type of immune cells (e.g., cytotoxic T cells, helper T cells, neutrophils, dendritic and antigen presenting cells, eosinophils, and mast cells) that localize to the site of infection.

The term “ligand” refers to a molecule that binds to a complementary receptor on a cell surface, and upon binding induces cellular downstream events.

An “agonist” is a molecule, compound, or drug that binds to physiological receptors and mimics the effect of the endogenous regulatory compounds. An agonist could be any molecule that mimics a biological activity of endogenous molecule, such as a chemokine. For example, an agonist, such as W-tide can mimic the activity of a chemoattractant. Agonists may also include small molecule compounds or antibodies.

An “antagonist” refers to any molecule that binds to a receptor and does not mimic, but interferes with, the function of the endogenous agonist. Such compounds are themselves devoid of intrinsic regulatory activity, but produce effects by inhibiting the action of an agonist (e.g. by competing for an agonist binding sites). Therefore, an antagonist is any molecule that partially or fully blocks, inhibits, or neutralizes a biological activity, such as cell migration or activation.

An “antigen” is a molecule that reacts with an antibody or T cell receptor or otherwise stimulates an immune response. An antigen is typically a peptide, a polypeptide, chemical compound, microbial pathogen, bacteria e.g., live, attenuated, or inactivated), a virus (including inactivated virus particles, modified live viral particles, and recombinant virus particles), a recombinant cell, glycoproteins, lipoproteins, glycopeptides, lipopeptides, toxoids, carbohydrates, tumor-specific antigens, and other immunogenic components of pathogens

A “chemoattractant” is a ligand to the chemoattractant receptor, that upon binding to the receptor induces cell migration.

The terms “W-peptide” or “W-tide” refer to high affinity ligands of FPRL1 receptor that are used in the methods and compositions of this invention and are able to modulate immune response in vivo and in vitro. Peptides, polypeptides, and/or proteins that include the W-tide amino acid sequence are also encompassed by the W-tides. W-tides also include all possible variants or fragments of the W-tide polypeptides.

Specific W-tides useful in accordance with the present invention are shown in Table 2. Standard amino acid abbreviations are used in the table below; lower case letters identify D-residues.

TABLE 2
Exemplary W-tide polypeptide sequences

| | |
|---|---|
| SEQ | |
| ID | |
| NO: | Amino acid sequence |

| | |
|---|---|
| 1 | His-Phe-Tyr-Leu-Pro-Met-CONH₂; HFYLPM |
| 2 | Met-Phe-Tyr-Leu-Pro-Met-CONH₂; MFYLPM |
| 3 | His-Phe-Tyr-leu-pro-D-Met-CONH₂; HFYLPm |
| 4 | Trp-Lys-Tyr-Met-Val-D-Met-CONH₂; WKYMVm |
| 5 | Trp-Lys-Gly-Met-Val-D-Met-NH₂; WKGMVm |
| 6 | Trp-Lys-Tyr-Met-Gly-D-Met-NH₂; WKYMGm |
| 7 | Trp-Lys-Tyr-Met-Val-Gly-NH₂; WKYMVG |
| 8 | Trp-kg-Tyr-Met-Val-D-Met-NH₂; WRYMVm |
| 9 | Trp-Glu-Tyr-Met-Val-D-Met-NH₂; WEYMVm |
| 10 | Trp-His-Tyr-Met-Val-D-Met-NH₂; WHYMVm |
| 11 | Trp-Asp-Tyr-Met-Val-D-Met-NH₂; WDYMVm |
| 12 | Trp-Lys-His-Met-Val-D-Met-NH₂; WKHMVm |
| 13 | Trp-Lys-Glu-Met-Val-D-Met-NH₂; WKEMVm |
| 14 | Trp-Lys-Trp-Met-Val-D-Met-NH₂; WKWMVm |
| 15 | Trp-Lys-Arg-Met-Val-D-Met-NH₂; WKRMVm |
| 16 | Trp-Lys-Asp-Met-Val-D-Met-NH₂; WKDMVm |
| 17 | Trp-Lys-Phe-Met-Val-D-Met-NH₂; WKFMVm |
| 18 | Trp-Lys-Tyr-Met-Tyr-D-Met-NH₂; WKYMYm |
| 19 | Trp-Lys-Tyr-Met-(Phe/Trp)-D-Met-NH₂; WKYM(F/W)m |
| 20 | Trp-Lys-Tyr-Met-Val-Glu-NH₂; WKYMVE |
| 21 | Trp-Lys-Tyr-Met-Val-Val-NH₂; WKYMW |
| 22 | Trp-Lys-Tyr-Met-Val-Arg-NH₂; WKYMVR |
| 23 | Trp-Lys-Tyr-Met-Val-Trp-NH₂; WKYMVW |
| 24 | Trp-Lys-Tyr-Met-Val-NH₂; WKYMV |
| 25 | Lys-Tyr-Met-Val-D-Met-NH₂; KYMVm |
| 26 | Lys-Tyr-Met-Val-NH₂; KYMV |
| 27 | Tyr-Met-Val-D-Met-NH₂; YMVm |
| 28 | Met-Val-D-Met-NH₂; MVm |

“W-tide polypeptide variant” means an active W-tide polypeptide having at least: (1) about 70% amino acid sequence identity with a W-tide sequence or (2) any fragment of a W-tide sequence. W-tide polypeptide variants include mutants of W-tides, or polypeptide fusions, modified polypeptides, or chemicals. For example, W-tide variants include W-tide polypeptides wherein one or more amino acid residues are added or deleted at the N- or C-terminus of the sequences of SEQ ID NOS: 1-28. A polypeptide variant will have at least about 90%, 91%, 92%, 93%, 94%, 95%, 96%, 97%, 98% amino acid sequence identity and most preferably at least about 99% amino acid sequence identity with W-tide polypeptide sequence. For example, W-tide of SEQ ID NO. 25 may be considered a variant of W-tide of SEQ ID NO 1, wherein the Glycine was substituted with Tyrosine. A more detailed discussion of alignment methodology and required conditions may be found in U.S. application Ser. No. 10/141,508, filed on May 7, 2002, which is incorporated by reference in its entirety, except that in the event of any inconsistent disclosure or definition from the present application, the disclosure or definition herein shall prevail.

“Percent (%) amino acid sequence identity” is defined as the percentage of amino acid residues that are identical with amino acid residues in a W-tide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Publicly available computer software such as BLAST, BLAST2, ALIGN2 or Megalign (DNASTAR) can be used to align polypeptide sequences. Parameters for measuring alignment, including any algorithms needed to achieve maximal alignment over the full length of the sequences being compared, can be determined.

In general, a W-tide polypeptide variant preserves W-tide polypeptide-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution, resulting in a “conservative variant.”

Changes in the amino acid sequence can be introduced by mutations that incur alterations in the amino acid sequences of the encoded W-tide that do not alter W-tide function. For example, amino acid substitutions at “non-essential” amino acid residues can be made in the sequence. A “non-essential” amino acid residue is a residue that can be altered from the original sequences of the W-tide without altering biological activity, whereas an “essential” amino acid residue is required for such biological activity. For example, amino acid residues that are conserved among the W-tide of the invention are predicted to be particularly non-amenable to alteration. Amino acids for which conservative substitutions can be made are well known in the art.

Useful conservative substitutions are shown in Table A, “Preferred substitutions.” Conservative substitutions whereby an amino acid of one class is replaced with another amino acid of the same type fall within the scope of the invention so long as the substitution does not materially alter the biological activity of the compound. If such substitutions result in a change in biological activity, then more substantial changes, indicated in Table B as exemplary, are introduced and the products screened for W-tide polypeptide biological activity.

TABLE A
Preferred substitutions

| | | Preferred |
|---|---|---|
| Original residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that affect (1) the structure of the polypeptide backbone, such as a β-sheet or α-helical conformation, (2) the charge (3) hydrophobicity, or (4) the bulk of the side chain of the target site can modify W-tide polypeptide function. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| Acidic | Asp, Glu |
| Basic | Asn, Gln, His, Lys, Arg |
| disrupt chain conformation | Gly, Pro |
| aromatic | Trp, Tyr, Phe |

W-tides can be produced by any method well known in the art, such as in vitro synthesis of peptides. In addition, expression via vectors such as bacteria, viruses and eukaryotic cells, may be also used. For example, the W-tides can be synthesized by the solid-phase method (Baek S H, 1996; and Seo J K, 1997). Briefly, peptides can be synthesized on a rapidamide support resin and assembled following the standard Fmoc/t-butyl strategy on an acid-labile linker. The composition of the peptides can be also confirmed by amino acid analysis known in the art (Baek S H, 1996).

W-tides can be either entirely composed of synthetic, non-natural analogues of amino acids, or a chimeric molecule of partly natural amino acids and partly non-natural analogs of amino acids. W-tides can also incorporate any amount of natural amino acid conservative substitutions. W-tide polypeptide compositions can contain any combination of non-natural structural components, which are typically from three structural groups: (a) residue linkage groups other than the natural amide bond (“peptide bond”) linkages; (b) non-natural residues; or (c) residues which induce secondary structural mimicry, i.e., inducing or stabilizing a secondary structure, e.g., a β turn, γ turn, β sheet, α helix conformation, and the like. Non-natural residues, as well as appropriate substitutions for each class of amino acids (Table B), are well known.

W-tides can be characterized by having all or some of its residues joined by chemical means other than natural peptide bonds. Individual peptide residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (Spatola, 1983).

Modified W-tides are also included in the scope of the present invention and can be generated by hydroxylation of proline and lysine; phosphorylation of the hydroxyl groups of seryl or threonyl residues; methylation of the α-amino groups of lysine, arginine and histidine; acetylation of the N-terminal amine; methylation of main chain amide residues or substitution with N-methyl amino acids; or amidation of C-terminal carboxyl groups.

W-tides can also include compositions that contain a structural mimetic residue, particularly a residue that induces or mimics secondary structures, such as a β turn, β sheet, α helix structures, γ turns, and the like. For example, substitution of natural amino acid residues with D-amino acids; N-α-methyl amino acids; C-α-methyl amino acids; or dehydroamino acids within a peptide can induce or stabilize β turns, γ turns, β sheets, or α helix conformations.

The variant W-tide nucleotide sequences can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce the W-tide variant DNA (Ausubel et al., 1987; Sambrook, 1989).

An “active” polypeptide or polypeptide fragment retains a biological and/or an immunological activity similar, but not necessarily identical, to an activity of the W-tide polypeptides shown in Table 2. Immunological activity, in the context of this immediate discussion of the polypeptide per se, and not an actual biological role for W-tide in modulating an immune response, refers to an aspect of a W-tide polypeptide in that a specific antibody against an antigenic epitope binds W-tide. Biological activity refers to a modulatory function, either inhibitory or stimulatory, caused by a W-tide polypeptide or polypeptide containing W-tide. A biological activity of W-tide polypeptide includes, for example, chemotaxis, modulating, inducing, enhancing, inhibiting or aiding an immune response.

Fusion polypeptides are useful in expression studies, cell-localization, bioassays, W-tide purification, and importantly in adjuvant applications when the peptide may be fused to the antigen(s) of interest. A W-tide “chimeric polypeptide” or “fusion polypeptide” comprises W-tide fused to a non-W-tide polypeptide. A non-W-tide polypeptide is not substantially homologous to W-tide of SEQ ID NOS: 1-28. A W-tide fusion polypeptide may include any portion to an entire W-tide, including any number of biologically active portions. In some host cells, heterologous signal sequence fusions may ameliorate W-tide expression and/or secretion.

Fusion partners can be used to adapt W-tide therapeutically. W-tide-Ig fusion polypeptides can be used as immunogens to produce anti-W-tide Abs in a subject, to purify W-tide ligands, and to screen for molecules that inhibit interactions of W-tide with other molecules. Additionally, fusions with antigens of interest can be used to facilitate vaccination/immunization procedures.

Fusion polypeptides can be easily created using recombinant methods. A nucleic acid encoding W-tide can be fused in-frame with a non-W-tide encoding nucleic acid, e.g., antigen(s) with which to immunize, to the W-tide NH₂— or COO— -terminus, or internally. Fusion genes may also be synthesized by conventional techniques, including automated DNA synthesizers. PCR amplification using anchor primers that give rise to complementary overhangs between two consecutive gene fragments that can subsequently be annealed and re-amplified to generate a chimeric gene sequence (Ausubel et al., 1987). Many vectors are commercially available that facilitate sub-cloning W-tide in-frame to a fusion moiety. Alternatively fusion polypeptides may be produced by synthetic methods well known in the art, such as solid phase peptide synthesis.

W-tides have certain properties when used as an adjuvant; namely, modulating an immune response. Other activities of the W-tides are known, including inducing chemotaxis on certain cells, including those expressing the formyl-peptide receptor-like-1 (FPRL1) receptor. In vitro chemotaxis (cell migration) assays can be used to identify W-tide chemotactic properties. Such assays physically separate the cells from the candidate chemoattractant using a porous membrane and assaying the cell migration from one side of the membrane to the other, indicating cell migration. As an example, a conventional cell migration assay, such as the ChemoTx® system (NeuroProbe, Rockville, Md.; (Goodwin, U.S. Pat. No. 5,284,753, 1994)) or any other suitable device or system (Bacon et al., 1988; Penfold et al., 1999) may be used. Cells expressing the target receptor are gathered. A candidate compound, such as W-tide peptides or other chemokine/chemokine-like compound is prepared, usually in a concentration series by serial dilution in a buffer. The concentration range is typically between 0.1 nM and 10 mM, but will vary with the compound being tested.

To start the cell migration assay, solutions of the various candidate compound concentrations are added to the lower chamber of a cell migration apparatus, and the cell suspension is placed into the upper chamber that is separated by a porous membrane (about 3 μm to about 5 μm, depending on cell type(s) and cell size(s)). The cells are incubated under culture conditions (about 37° C. for human cells) for 60 to 180 minutes in a humidified tissue culture incubator. The incubation period depends on the cell type and if necessary, can be determined empirically.

After terminating the assay, non-migrating cells on the upper chamber of the apparatus are removed using a rubber scraper or other manual method, enzymatically or chemically, e.g., EDTA and EGTA solutions. The membrane that separates the two chambers is then removed from the apparatus and rinsed with Dulbecco's phosphate buffered saline (DPBS) or water. The number of cells that migrated into the lower chamber is then determined. Cell migration at levels above background (without a chemotactic or candidate compound), indicate that the candidate compound is chemotactic for the tested cells.

A candidate compound, such as W-tide is considered chemotactic for a particular cell type if, at a concentration of about 1 pM to about 1 μm (e.g., between about 1 nM and 500 nM, e.g., 1 nM, about 10 nM, about 100 nM, or between about 1 pg/ml and about 10 μg/ml, e.g., between about 1 ng/ml and 1 μg/ml, e.g., about 10 ng/ml, about 100 ng/ml or about 1 μg/ml) attracts the cell at least 2-fold to 8-fold or more than a negative control.

Chemotactic properties of a W-tide can be determined in animals, e.g., mammals such as non-human primates and mice. In one in vivo assay, the W-tide (e.g., 1-100 μg in PBS) is administered by sub-cutaneous injection. After about 24 to about 96 hours or more, the presence or absence of cell infiltration is determined, using routine histological techniques. If an infiltrate is present, the cells are identified by type (mononuclear, neutrophil, dendritic, etc.) and are quantified.

The invention provides for both prophylactic and therapeutic methods of treating a subject at risk of (or vulnerable to) a disorder or having a disorder associated with FPRL1 receptor or FPRL1 ligand activity. Examples include previously discussed AD, CJD, and SLE.

For prophylactic use, compositions containing W-tides are administered (e.g., in conjunction with antigens) to a subject. For therapeutic use, compositions containing the W-tides are administered to a subject once a disease is detected, diagnosed or even treated, such as after surgical removal of a tumor.

According to one embodiment of this invention, W-tides or conservative variants, fragments, etc. may be administered in compositions, such as those used to modulate an immune response; one or more of the W-tides may be included.

The compositions also include antigens of interest. W-tide polypeptides may also be associated (covalently or non-covalently) to the antigen of interest. In some embodiments, the W-tides and antigens are administered simultaneously. In other embodiments, the W-tides are administered in sequence with conventional adjuvants and pharmaceutical carriers containing antigens. In yet other instances, W-tides in either form may be administered prior to or after administration of the antigen. When W-tide compositions are administered separately from antigen compositions, the compositions are administered at the same physical location in a subject. Mixtures of two or more antigens may be used. The antigen may be purified. The antigen is distinct from the W-tide used in the composition.

Exemplary antigens or vaccine components of the invention include antigens derived from microbial pathogens such as bacteria [e.g., Pertussis (Bordetella pertussis, inactivated whole organism); Anthrax (Bacillus anthraxis, protective antigen) Cholera (Vibrio cholerae, whole killed organism); Meningitis (Neisseria meningitidis, polysaccharide from organism); Lyme Disease (Borrelia burgdorferi, lipoprotein OspA); Haemophilus B (Haemophilus influenza B polysaccharide, Tetanus conjugate or OmpC); Pneumonia (Streptococcs pneumoniae capsular polysaccharide) Typhoid (Salmonella typhi polysaccharide vaccine, killed whole organism)], viruses including inactivated virus particles, modified live viral particles, and recombinant virus particles to Influenza virus; Smallpox, Hepatitis A; Hepatitis B; Hepatitis C; Measles; Rubella virus; Mumps; Rabies; Poliovirus; Japanese Encephalitis virus; Rotavirus; Varicella], Diphtheria (Corynebacterium diphtheriae), Tetanus (Clostridium tetani), Malaria, and fungal antigens.

In one aspect, the present invention provides a method of modulating, for example, by eliciting or enhancing an immune response to an antigen, e.g., a predetermined or specified antigen. In some embodiments the antigen is linked to a protein carrier. For example, a W-tide and an antigen may be physically linked, such as by a fusion protein, chemically cross-linking or complexes such as biotin and streptavidin.

In another aspect, the method of the invention involves administration of an immunogen (a substance that induces a specific immune response), in addition to a W-tide composition and an antigen.

In one aspect, while a monomeric W-tide may be sufficient to interact with FPRL1 and thereby modulate a cellular response, multimeric synthetic ligands can have far greater ability to interact with FPRL1 and thereby modulate a cellular response. The term “multimeric” refers to a presence of more than one units of ligand linked together, for example several individual molecules of W-tide, conservative variants or fragments thereof. Therefore, multimeric W-tide compositions can also be administered according to the methods of this invention.

The invention is used to provide protection from exogenous foreign infectious pathogenic agents prior to exposure. In addition, the invention can be used to provide therapeutic effects against exogenous foreign pathogens to which an individual has been exposed or to individual displaying symptoms of exposure.

The invention can be used to treat cancers, including, but not limited to, melanomas, lung cancers, thyroid carcinomas, breast cancers, renal cell carcinomas, squamous cell carcinomas, brain tumors and skin cancers. For example, the antigen may be a tumor-associated antigen (tumor specific-antigen). Tumor antigens are molecules, especially cell surface proteins, which are differentially expressed in tumor cells relative to non-tumor tissues.

W-tide compositions can be administered to tumors by for example, injection into a solid tumor to elicit an immune response to cancer cells, or injection in tissue surrounding a solid tumor, e.g., within 2 cm, of a solid tumor. Without intending to be bound by a particular mechanism, it is believed that W-tides modulate an immune reaction to the endogenous (e.g., tumor) antigen by recruiting immune cells to the site of administration.

To modulate, especially promote an immune response to tumors and cancers, W-tide compositions may be administered at the sites of abnormal growth or directly into the tissue (i.e., a tumor). Tumor or cancer antigens may then detected by the W-tide-recruited or activated leukocytes, such as monocytes cells. By modulating an immune response to these antigens, tumors and cancers could be attacked by the body and are reduced or eliminated. As such, these methods represent treatments for conditions involving uncontrolled or abnormal cell growth, e.g., tumors and cancers. Immune responses to tumors and cancers may also be promoted and/or modulated by administering isolated polypeptide tumor antigens with W-tides. W-tides may either be conjugated to the antigen or unconjugated.

W-tide compositions may contain a conventional adjuvant. Conventional adjuvants typically convert soluble protein antigens into particulate material. Conventional adjuvants include Freund's incomplete, Freund's complete, Merck 65, AS-2, alum, aluminum phosphate, mineral gels such as aluminum hydroxide, and surface active substances such as lysolecithin, pluronic polyols, polyanions, peptides, oil emulsions, keyhole limpet hemocyanin, dinitrophenol, and other suitable adjuvants. Other useful adjuvants include, but are not limited to, bacterial capsular polysaccharides, dextran, IL-12, GM-CSF, CD40 ligand, IFN-γ, IL-1, IL-2, IL-3, IL-4, IL-10, IL-13, IL-18 or any cytokine or bacterial DNA fragment. Furhermore, commercially available CpG oligonucleotides may be used as adjuvants. CpG oligonucleotides are short synthetic oligonucletides (DNA-like sequences) that invoke potent innate and adaptive immune responses of the body's immune system, comprising of both antibody- and cell-mediated pathways.

The W-tide, the antigen, or both may be delivered as polynucleotides, such that the polypeptides are generated in situ. In the case of naked polynucleotides, uptake by cells can be increased by coating the polynucleotide onto a carrier, e.g. biodegradable beads, which is efficiently transported into cells. In such vaccines, the polynucleotides may be present within any of a variety of delivery systems, including nucleic acid expression systems, bacterial and viral expression systems.

Vectors, used to shuttle genetic material from organism to organism, can be divided into two general classes: Cloning vectors are replicating plasmid or phage with regions that are non-essential for propagation in an appropriate host cell and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA, such as W-tide. In expression vectors, the introduced DNA is operably-linked to elements such as promoters that signal to the host cell to transcribe the inserted DNA. Nucleic acid is “operably-linked” when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation.

Inducible promoters that control gene transcription in response to specific factors can be exceptionally useful. Operably-linking a W-tide and/or antigen polynucleotide to an inducible promoter can control the expression of a W-tide and/or antigen polypeptide or fragments. Examples of classic inducible promoters include those that are responsive to α-interferon, heat shock, heavy metal ions, and steroids such as glucocorticoids (Kaufman, 1990), and tetracycline. Other desirable inducible promoters include those that are not endogenous to the cells in which the construct is being introduced, but are responsive in those cells when the induction agent is exogenously supplied. In general, useful expression vectors are often plasmids. However, other forms of expression vectors, such as viral vectors (e.g., replication defective retroviruses, adenoviruses and adeno-associated viruses) are contemplated.

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences.

W-tide compositions may contain one or more antigens or antigen-encoding polynucleotides. Antigens can be administered in combination with W-tides (i.e., in the same mixture). Alternatively, they can be administered separately. In one aspect, the invention provides an immunization method in which a combination of one or more antigens (or antigen-encoding polynucleotides) and one or more W-tides are administered to a subject. The antigen or W-tide may be administered in a delivery vehicle such as a physiologically acceptable excipient.

The antigen may be administered simultaneously with the W-tide composition or the antigen and the W-tide composition is administered at different times, typically to the same site. Preferably, the W-tide composition is administered simultaneously with the antigen. If administered at different times, the chemotactic composition (without the antigen) can be administered, for example, between about 15 minutes and about 96 hours prior to the administration of the antigen, more often between about 15 minutes and about 48 hours, more often between 24 hours and 48 hours, prior to the administration of the antigen.

When a W-tide composition and an antigen composition are injected at the same site in a subject, preferably the injections are within 2 cm of each other, preferably within 1 cm or preferably within 0.5 cm of each other on the two dimensional surface of the body. The administrations should also be done to a similar depth and to the same tissue layers. For intramuscular injections, the depth should be more precisely monitored to achieve a three dimensional equivalent placement of the W-tide and the antigen to within 2 cm of each other, preferably to within 1 cm, and more preferably to within 0.5 cm. The injection site can be marked with an indelible ink to assist the physician.

One dose (administration) of the composition may be given. However, the first administration may be followed by boosting doses. For example, the W-tide composition is administered in multiple doses, often in combination with an antigen (e.g., by co-administration). The W-tide composition (optionally including antigen) may be administered once, twice, three times, or more. The number of doses administered to a subject is dependent upon the antigen, the extent of the disease, and the response of a subject to the W-tide composition. Within the scope of the present invention, a suitable number of doses include any number required to immunize a subject to a predetermined antigen.

A second administration (booster) of the W-tide composition and antigen may be given between about 7 days and 1 year after the first administration. The time between the first and second administrations may be 14 days to 6 months, 21 days and 3 months, often between about 28 days and 2 months after the original administration. A third administration (second booster) may be given between about 14 days and 10 years after the first administration, e.g., between about 14 days and 3 years, often between about 21 days and 1 year, very often between about 28 days and 6 months after the first administration. Subsequent boosters may be administered at 2 week intervals, or 1 month, 3 month or 6 month to 10 year intervals.

A variety of vaccine administration doses and schedules can be developed easily; the determination of an effective amount and number of doses of W-tides of the invention, antigens, or some combination of W-tides and antigens for administration is also well within the capabilities of those skilled in the art.

Typically, the amount of W-tide and antigen will be administered to a subject that is sufficient to immunize a subject against an antigen (i.e., an “immunologically effective dose” or a “therapeutically effective dose”). An amount adequate to accomplish an “immunologically effective dose” will depend in part on the W-tide and antigen composition, the manner of administration, the stage and severity of the disease being treated, the weight and general state of health of the subject, and the judgment of the prescribing physician or other qualified personnel.

The effective dose of antigen and W-tide can be formulated in animal models to achieve an induction of an immune response; such data can be used to readily optimize administration to humans based on animal data (see Examples). A dose of W-tide polypeptide will typically be between about 1 fg and about 100 μg, often between about 1 pg and about 100 μg, more often between about 1 ng and about 50 μg, and usually between about 100 ng and about 50 μg. In some embodiments, the dose is between about 1 fg and about 100 μg per kg subject body weight, often between about 1 pg and about 100 μg, more often between about 1 ng and about 50 μg, and usually between about 100 ng and about 50 μg per kg subject body weight.

The amount of antigen will vary with the identity and characteristics of the antigen. A W-tide composition may contain one or more antigens and one or more W-tides at a molar or weight ratio of about 1:1000 or greater, W-tide to antigen. Other useful ratios are between about 1:10 and 1:1000, between about 1:10 and 1:1000, or greater than 1:1000. The ratio of antigen to W-tide in the composition may vary between about 1:10 and 10:1.

In order to be useful as a biotechnological tool or component to a prophylactic or therapeutic agent, it is desirable to provide a peptide agent in such form or in such a way that a sufficient affinity for FPRL1 or FPR is obtained. While a monomeric peptide agent may be sufficient to interact with FPRL1 and thereby modulate a cellular response, multimeric synthetic ligands can have far greater ability to interact with FPRL1 and thereby modulate a cellular response.

The W-tide-containing compositions of the invention may be administered in a variety of ways and in various forms. The W-tide composition may include carriers and excipients. These carriers and excipients for use in the body, (i.e. for prophylactic or therapeutic applications) are desirably physiological, non-toxic, and preferably non-immunosuppresive. Suitable carriers and excipients for use in the body include appropriate buffers, carbohydrates, mannitol, proteins, polypeptides or amino acids such as glycine, antioxidants, bacteriostats, chelating agents, suspending agents, thickening agents and/or preservatives; water, oils, saline solutions, aqueous dextrose and glycerol solutions, other pharmaceutically acceptable auxiliary substances as required to approximate physiological conditions, such as buffering agents, tonicity adjusting agents, wetting agents, etc.

Other convenient carriers include multivalent carriers, such as bacterial capsular polysaccharide, a dextran or a genetically engineered vector. In addition, W-tides and/or antigens are prepared with carriers that protect the compound against a rapid elimination from the body, such as sustained-release formulations, including implants and microencapsulated delivery systems. Biodegradable or biocompatible polymers can be used, such as ethylene vinyl acetate, polyanhydrides, polyglycolic acid, collagen, polyorthoesters, and polylactic acid. Polyethylene glycols, e.g. PEG, are also good carriers. Such materials can be obtained commercially from ALZA Corporation (Mountain View, Calif.), and NOVA Pharmaceuticals, Inc. (Lake Elsinore, Calif.), or prepared by one of skill in the art. These materials allow for the release of W-tides and/or antigens over extended periods of time, such that without the sustained release formulation, the W-tides and/or antigens would be cleared from a subject's system or degraded.

While any suitable carrier may be used to administer the compositions of the invention, the type of carrier will vary depending on the mode of administration. Compounds may also be encapsulated within liposomes. Biodegradable microspheres are convenient in some instances as carriers; for example, such as those described in (Tice et al., U.S. Pat. No. 5,942,252, 1999).

A suitable conventional adjuvant may also be incorporated into the composition.

The W-tide compositions of the invention may be administered in a variety of ways, including by injection (e.g., intradermal, subcutaneous, intramuscular, intraperitoneal etc.), by inhalation, by topical administration, by suppository, by using a transdermal patch or by mouth.

When administration is by injection, compositions may be formulated in aqueous solutions, preferably in physiologically compatible buffers such as Hanks solution, Ringer's solution, or physiological saline buffer. The solution may contain formulatory agents such as suspending, stabilizing and/or dispersing agents. Alternatively, the chemotactic composition may be in powder form for constitution with a suitable vehicle, e.g., sterile pyrogen-free water, before use.

Inhalation-delivered compositions may be as aerosol sprays from pressurized packs or a nebulizer with the use of a suitable propellant, e.g., dichlorodifluoromethane, trichlorofluoromethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the proteins and a suitable powder base such as lactose or starch.

Systemic administration can also be transmucosal or transdermal. For transmucosal or transdermal administration, penetrants that can permeate the target barrier(s) are selected. Transmucosal penetrants include detergents, bile salts, and fusidic acid derivatives.

For topical administration, the compositions may be formulated as solutions, gels, ointments, creams, suspensions, and the like, as are well known in the art. In some embodiments, administration is by means of a transdermal patch. Suppository compositions may also be formulated to contain conventional suppository bases.

When administration is oral, a composition can be readily formulated by combining the composition with an inert diluent or edible and/or pharmaceutically acceptable carriers. Solid carriers include mannitol, lactose, magnesium stearate, etc.; such carriers enable the formation of tablets, pills, dragees, capsules, liquids, gels, syrups, slurries, suspensions etc., for oral ingestion. Such formulations may be powders, capsules and tablets; suitable excipients include fillers such as sugars, cellulose preparation, granulating agents, and binding agents.

Sterilization of the compositions is desirable, such as that accomplished by conventional techniques or sterile filtering. The resulting aqueous solutions may be packaged for use as is, or lyophilized. Additionally the compositions may be prepared by GMP techniques.

Nucleic acid molecules, such as those encoding W-tides, can be inserted into vectors and used as gene therapy vectors for genetic vaccination or ‘prime-boost’ vaccination regimes. A ‘prime-boost’ vaccination is a type of vaccination where administration of a genetic vaccine (such as a recombinant vector vaccine) is followed by a second type of vaccine (such as a protein subunit vaccine). The goal of ‘prime-boost’ vaccination is to stimulate different kinds of immune responses and enhance the body's overall immune response. Gene therapy techniques have recently become quite advanced and are meeting enviable success (Meikle, 2002). Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

By “antibody” is meant a monoclonal or a polyclonal antibody per se, immunologically effective fragments thereof (e.g., F_{ab}, F_{ab′}, or F_{(ab′)2}), or a single chain version of the antibodies, usually designated as Fᵥ regions. Methods of producing polyclonal and monoclonal antibodies, including binding fragments and single chain versions are well known in the art. However, many antigens are incapable of triggering an adequate antibody response. In one embodiment, a composition comprising a W-tide of the invention and an antigen is administered to a subject, thus modulating the immune response in the subject.

To elicit antibodies, in one embodiment, the W-peptide and the antigen can be co-administered. In another embodiment, the W-peptide and the antigen are administered separately. In both types of the administration, the antibody titer to an antigen is increased preferably by at least two fold.

Polyclonal or monoclonal antibodies are subsequently prepared by standard techniques.

In another aspect, the compositions of the invention are administered to a subject to modulate the innate immune response. The innate immune response is the body's initial defense against pathogens and is elicited by a variety of cells including APCs. These cells express surface and cytoplasmic receptors that recognize molecules of foreign origin (e.g., bacterial and viral nucleic acids, proteins, carbohydrates). Upon detecting these signals, the dendritic cells and macrophage elicit a defensive response that includes the release of cytokines (including interferons, TNF-α, and IL-12) and chemokines that attract cells such as immature dendritic cells, macrophage, NK cells, and granulocytes, to the site of challenge.

The compositions of the invention can be used to attract dendritic cells and other cells to the site of administration, but also to modulate these cells into eliciting elements of the innate immune response to confer non-specific protection while the body is generating the adaptive response. For example, a W-tide composition is administered prior to or post exposure of an anticipated infection, including those that are sinisterly applied, such as in bioterrorism. In another embodiment, W-tides are administered with “foreign” molecules (e.g., bacterial or viral nucleic acids, proteins, carbohydrates, or synthetic elements which mimic these elements).

The following examples are given to illustrate the invention and are not meant to limit it in any way.

### EXAMPLES

### Example 1 Methods

Unless stated otherwise, reagents are obtained from Sigma Chemical Co. (St. Louis, Mo.).

W-Tide (SEQ ID NO: 4) Peptide Preparation

The peptide of SEQ ID NO: 4, “W-tide”, is chemically synthesized and purified (Phoenix Pharmaceuticals; Belmont, Calif.). The material is suspended in phosphate-buffered saline (PBS) at a concentration of approximately 1 mg/ml and stored at −20° C.

Enzyme-Linked Immunosorbent Assays (ELISAs)

First, 96-well U-bottom plastic dishes are coated overnight with about 0.1 to 1 μg anthrax recombinant protective antigen (PA) in 100 μl PBS per well. The next day, the dishes are rinsed with PBS, blocked with PBS containing 5% fetal bovine serum (FBS), and rinsed with PBS again. Plasma samples from experimental animals (see below) are diluted 10¹- to 10⁵-fold and added to the dishes for 2 hours, after which the dishes are again rinsed with PBS. The dishes are then incubated with biotinylated goat anti-mouse IgG detection antibodies, then rinsed with PBS and incubated with streptavidin-linked horseradish peroxidase (SA-HRP). After a final rinsing with PBS, the HRP substrate 2,2′-Azinobis [3-ethylbenzothiazoline-6-sulfonic acid]-diammonium salt is added. Color development is measured with an ELISA plate reader at 405 nm, and optical density (OD) units are converted to arbitrary “antibody units,” where a unit is defined as the inverse of the plasma dilution that produces 50% of the maximum response from a standard curve obtained by serial dilution of an ascites collected from PA-injected mice and containing PA-specific antibodies.

Dendritic Cell Purification

Substantially purified dendritic cells (including subpopulations of mature or immature cells) are prepared. Subpopulations of dendritic cells include: (1) immature peripheral blood monocyte derived cells, (2) mature peripheral blood monocyte derived cells, and (3) cells derived from CD34-expressing precursors.

Human or macaque dendritic cells of various developmental stages can be generated in culture from CD14-expressing blood progenitors using specific cytokines. A separate lineage of dendritic cells can be differentiated from CD34-expressing precursor cells from cord blood or bone marrow. Finally, immature and mature dendritic cells from peripheral blood mononuclear cells (PMBCs) can also be produced (Bender et al., 1996). Mature dendritic cells can be made using macrophage conditioned medium and double stranded RNA-poly (I:C) stimulation (Cella et al., 1999; Romani et al., 1996; Verdijk et al., 1999).

To confirm that a population of dendritic cells has been isolated, marked changes in chemokine receptor expression during dendritic cell maturation can be used to identify and confirm cell stage (Campbell et al., 1998; Chan et al., 1999; Dieu et al., 1998; Kellermann et al., 1999). For example, produced mature dendritic cells can be characterized by using cellular markers and fluorescence-activated cell sorting (FACS). Generated dendritic cells express higher levels of MHC class II on the cell surface than immature dendritic cells. Expression of CD80, CD83 and CD86 are also up-regulated. Chemokine receptor expression also changes dramatically during maturation; e.g., CCR1 and CCR5 are down-regulated in mature cells while CCR7 is up-regulated. Functional characteristics may also be exploited to confirm a cell type. For example, mature dendritic cells are incapable of taking up antigen efficiently, but gain the ability to stimulate the proliferation of naive T cells and B cells. Mature dendritic cells also change their migratory behaviors, being unresponsive to CCR1, CCR2 and CCR5 ligands while being newly responsive to CCR7 ligands.

### Example 2 W-Tide (SEQ ID NO: 4) Attracts Dendritic Cells

This example describes an in vivo assay in which the ability of two chemokines and W-tide (SEQ ID NO: 4) to attract dendritic cells is demonstrated.

The following chemokines are obtained from R&D Systems (Minneapolis, Minn.): mC10, and GM-CSF.

The following peptides are synthesized at Phoenix Pharmaceuticals (San Carlos, Calif.): W-tide (SEQ ID NO: 4), control peptide (SEQ ID NO: 29, Gly Ala Ala His Ser Leu Thr Met Gln Pro Gly Ile Lys Arg Arg Trp Leu Met), W-tide randomly conjugated to PA in either a 1:1 or 1:4 ratio (by MBS coupling method), and W-tide conjugated to PA at the C-terminus (C-term, made by the addition of a cysteine), and W-tide variant (SEQ ID NO: 25).

In three separate experiments, chemokines or peptides (1 μg or 10 μg in PBS) are injected intradermally into BALB/c or C57BI/6 mice (Jackson Laboratory; Bar Harbor, Me.). In each experiment, one mouse receives an injection of PBS only as a negative control. At various times after injection, the mice are euthanized, and the area around the injection site is excised and subjected to immunohistology. Frozen sections are stained with anti-DEC-205 antibody (Bio-Whittaker Molecular Applications; Rockland, Me.) that recognizes a dendritic cell-specific molecule (Kraal et al., 1986). A relative staining number on a scale of 0 to 5 is assigned to each section (0, none; 1, slight; 2, mild; 3, moderate; 4, severe).

As shown in Tables 3 and 4, mC10, W-tide without an antigen (SEQ ID NO: 4), and W-tide variant without an antigen (SEQ ID NO: 25), shows excellent infiltration of DEC-205-labeled cells. Similar effect is expected for W-tide with the antigen.

TABLE 3
Dendritic cell infiltration in BALBc mice (various doses)

| | | Time | |
|---|---|---|---|
| Polypeptide | Dose | (hours) | Score |
| Saline | 0 μg | 6 | 0 |
| | | | 1 |
| | | | 0 |
| | | 30 | 2 |
| | | | 1 |
| | | | 2 |
| mC10 | 1 μg | 6 | 2 |
| | | | 2 |
| | | | 2 |
| | | 30 | 2 |
| | | | 2 |
| | | | 3 |
| | 10 μg | 6 | 2 |
| | | | 2 |
| | | | 2 |
| | | 30 | 3 |
| | | | 1 |
| | | | 1 |
| W-tide | 1 μg | 6 | 2 |
| (SEQ ID NO: 4) | | | 2 |
| | | | 3 |
| | | 30 | 3 |
| | | | 2 |
| | | | 3 |
| | 10 μg | 6 | 2 |
| | | | 2 |
| | | | 3 |
| | | 30 | 3 |
| | | | 0 |
| | | | 1 |

TABLE 4
Infiltration in BALB/c mice, various doses

| | Time | |
|---|---|---|
| Polypeptide | (hours) | Score |
| Saline | 6 | 1 |
| | | 2 |
| | | 1 |
| Saline | 30 | 1 |
| | | 1 |
| | | 1 |
| W-tide variant (SEQ ID NO: 25) | 30 | 3 |
| | | 2 |
| | | 1 |
| W-tide (SEQ ID NO: 4) | 30 | 0 |
| | | 2 |
| | | 2 |
| W-tide and PA | 30 | 3 |
| | | 3 |
| | | 1 |
| PA-W-tide C-term | 30 | 2 |
| | | 2 |
| | | 2 |
| PA-W-tide 1:1 | 30 | 4 |
| | | 4 |
| | | 3 |
| PA-W-tide 1:4 | 30 | 3 |
| | | 3 |
| | | 4 |
| Control peptide | 30 | 1 |
| | | 0 |
| | | 2 |

### Example 3 W-Tide (SEQ ID NO: 4) Induces Mononuclear Cell Infiltration

Different amounts (0,1, or 10 μg in 100 μl PBS) of W-tide (SEQ ID NO: 4), and mC10 polypeptides (see Table 5) are injected subcutaneously in BALB/c mice under anesthesia on days 0 and 14. Twenty-four and 48 hours post first injection, 6 mm skin punch biopsies are taken using aseptic technique and then bisected. One portion of the biopsy is embedded in OCT compound, flash frozen in liquid nitrogen and stored at −70° C. The other portion is immersed in formalin and embedded in paraffin wax; subsequently, sections cut on a microtome are stained with hematoxylin and eosin and then microscopically examined for cell infiltration into the dermis (Table 5). As a negative control, mice are injected with PBS (saline) lacking any polypeptides.

Mononuclear cell infiltration is scored on a scale of 0 to 5: 0, very mild perivascular mononuclear inflammatory infiltration throughout the dermis; 1, a mild perivascular mononuclear inflammatory infiltrate seen throughout the dermis; 2, a mild/moderate perivascular mononuclear inflammatory infiltrate seen throughout the dermis; 3, a moderate perivascular mononuclear inflammatory infiltrate seen throughout the dermis; 4, an extensive perivascular mononuclear inflammatory infiltrate seen throughout the dermis; 5, a florid perivascular mononuclear inflammatory infiltrate seen throughout the dermis. Intermediate scores are indicates, e.g., “2/3” represents a score between 2 and 3.

It is expected that W-tide without an antigen at 10 μg will cause a moderately strong infiltration in the animals. The 10 μg administration may cause more infiltration than the 100 μg or 1 μg administration. When lower chemokine concentrations are used, mC10 will cause little to no infiltration in this experiment.

### Example 4 Procedure to Determine the Chemotactic Properties of a Candidate Molecule

To perform chemotaxis assays, 29 μl of a W-tide or known chemoattractants for a specific cell type, such as L1.2 cells expressing FPRL1 or other FPRL1 expressing cells, at 0, 1, 10 and 100 nM are placed in the wells of the lower chamber of a 96-well chemotaxis chambers (Neuroprobe; Gaithersburg, Md.). Day 7 immature dendritic cells are harvested, washed once with chemotaxis buffer (0.1% BSA in Hank's balanced salt solution (HBSS; Invitrogen, Carlsbad, Calif.), with Ca⁺⁺ and Mg⁺⁺), and resuspended in chemotaxis buffer at 5×10⁶ cells/ml. Twenty microliters of cells is placed onto the filter. The chambers are incubated for 90 minutes at 37° C. Migration is terminated by removing non-migrating cells on the top of the filter using a rubber scraper. After removing the filter and rinsing with Dulbecco's phosphate buffered saline (DPBS; Hyclone, Darra, Queensland, Australia), cells that have migrated are quantified by cell staining, such as the Hema3 staining kit (Fisher Scientific; Tustin, Calif.) or the CyQuant assay (Molecular Probes; Eugene, Oreg.), a fluorescent dye method that measures nucleic acid content and microscopic observation. The lower chamber is inspected microscopically to determine if any cells have migrated into the wells.

If significant number of cells is present in the wells, quantification is done in the wells as well as the filter. The magnitude of migration is calculated as the ratio of absorbance between the wells with chemoattractants and the wells with chemotaxis buffer alone.

### Example 5 Identification of Infiltrating Cells

To better define the identity of the infiltrating cells seen in Example 3, the same samples are analyzed by immunohistochemistry using antibodies specific for different cell types. These antibodies include: CD68 (expressed on macrophages, neutrophils and dendritic cells), MHC II (antigen-presenting cells, e.g. macrophages and dendritic cells), HAM-56 (macrophages), fascin (dendritic cells, endothelial cells and epithelial cells), elastase (neutrophils), cytokeratin (epithelial cells), CD3 (T cells), CD20 (B cells), and CD1a (Langerhans cells).

The mC10-injected skin samples will contain primarily antigen-presenting cells, including macrophages and dendritic cells, but few neutrophils. The W-tide injected skin samples will contain primarily monocytes, neutrophils and dendritic cells; no T-cells are stimulated.

### Example 6 W-Tide (SEQ ID NO: 4) Adjuvant Activity in BALC/c Mice

Since the W-tides recruit APCs, including dendritic cells, to the site of injection, these polypeptides are tested for their ability to act as immunization adjuvants to augment the immune response to a co-injected foreign antigen. Seven groups of mice, 5 mice per group, are injected subcutaneously with anthrax recombinant protective antigen (rPA) as an antigen.

The first group of mice receives rPA alone.

The second group receives rPA with 1 μg of W-tide.

The third group receives rPA and 10 μg of W-tide.

The fourth group receives 1 μg of W-tide alone.

The fifth group receives 10 μg of W-tide alone.

The formulations (containing 2.5 μg rPA and varying μg of W-tide polypeptide) are injected subcutaneously in 100 μl at days 0 and 14, following again with a final boost of 2.5 μg of rPA alone on day 21. 100 ul of periorbital blood is drawn from each mouse on days 0, 14 and 21, and the blood samples are then subjected to centrifugation to clarify the plasma. The plasma supernatant is analyzed by sandwich ELISA to determine the levels of anti-PA antibodies using PA-coated plastic dishes and a biotinylated anti-mouse IgG detection antibody.

This experiment will show that W-tide when administered with an antigen (PA) causes a significantly greater induction of anti-PA antibodies in mice, as compared to administration of the antigen alone, or W-tide alone at various concentrations.

Furthermore, to perform the recall assays of cellular response, spleens are harvested and blood collected by cardiac puncture on day 27 at animal sacrifice. These spleens are then dissociated into cell culture in 5 ml of DMEM +10% FBS. The splenocytes are counted and plated in 96 well round bottom plate in triplicate at approximately 4×10⁵ cells/well. These cell cultures are then treated with either the media (DMEM+10% FBS) alone, Concavalin A (Sigma, MO), 10 μg of rPA. These plates are next incubated at 37° C. in 5% CO₂ incubator. Five days post-plating, the cell cultures are treated with the media containing 50 mCi/ml ³H thymidine and the plates are further incubated for 18 hours at 37° C. Following this incubation, the cells were harvested by freeze/thaw method. Briefly, 96 well plates are transferred to −80 C for 1 hour to lyse cells. Plates are removed and placed at 37° C. for 1 hour. Cells are harvested by vacuum onto water wetted glass filter plates and retained counts quantified by scintillation counting. Retained counts in the PA stimulated samples are compared with media alone (background) and 5 ug/ml Concavalin A induced (positive control) samples to determine the degree of PA induced proliferation.

This experiment will demonstrate that when an antigen (PA) is administered to the mice together with the W-tide, antigen-specific anti-PA lymphoproliferation in mice spleen cells is significantly greater than that of mice receiving administrations of either PA alone or W-tide alone.

### Example 7 Procedure to Evaluate W-Tide in Augmenting or Modulating Systemic and/or Mucosal Immune Responses to Infectious Diseases

Groups of mice are injected either subcutaneously, intradermally, intranasally, or by any other mode with varying doses of the virus, bacterium, or parasite under study, using a typical immunization schedule, e.g., days 0, 7, and 14, in the presence or absence of W-tide given simultaneously with the microorganism in an appropriate formulation which may include adjuvants. Serum and/or mucosal secretions are collected on days −7, 0, 7, 14, 21, 28 and 35 for antigen-specific antibody analysis by ELISA. Mice are sacrificed at different time intervals (such as after the last immunization to quantitate the antigen-specific antibody-forming cells and antigen-specific T cell responses (both cytotoxic and helper T cell populations)) present in immune compartments, using standard procedures.

### Example 8 Procedure to Evaluate W-Tide in Augmenting or Modulating Anti-Tumor Immunity in Cancer Immunotherapy Regimens

While many tumor cells express unique tumor-associated antigens, these antigens are invariably weak immunogens and fail to generate potent anti-tumor immunity during tumor progression. The ability of W-tide, to augment protective anti-tumor immunity can be evaluated using a model system of cancer immunotherapy in mice. In this model, mice are transplanted with a syngeneic thymoma (EL4 cells; American Type Tissue Collection (ATTC); Manassas, Va.; no. TIB-39) that have previously been transfected with the experimental protein antigen PA. Without further intervention, the tumor grows and eventually kills the mouse. Animals can be at least partially protected by vaccinating them with PA formulated with W-tide to induce an antigen-specific immune response directed against the PA-transfected thymoma cells. This model is effective to evaluate the relative efficacy of W-tide and other adjuvants in augmenting or modulating protective anti-tumor immunity. Positive controls in this model may include the following adjuvants: CFA, IFA, alum and GM-CSF. The ability of W-tide to augment cancer immunotherapy regimens can be evaluated by comparison to these known adjuvants.

### Example 9 Procedure to Evaluate Ability of W-Tide to Modulate Allergen-Specific Immune Responses to Decrease Allergen-Induced Pathology

An animal model of asthma can be induced by sensitizing rodents to an experimental antigen (e.g., OVA) by standard immunization, and then subsequently introducing that same antigen into the rodent's lung by aerosolization. Three series of rodent groups, comprising 10 rodents per group, are actively sensitized on Day 0 by a single intraperitoneal injection with 2.5 μg PA in phosphate-buffered saline (PBS), along with an IgE-selective adjuvant, such as aluminum hydroxide (“Alum” adjuvant). At 11 days after sensitization at the peak of the IgE response, the animals are placed in a Plexiglas chamber and challenged with aerosolized OVA (1%) for 30 minutes using an ultrasonic nebulizer (De Vilbliss Co.; Somerset, Pa.). One series of mice additionally receives phosphate buffered saline (PBS) and Tween 0.5% intraperitoneally at the initial sensitization and at different dosing schedules thereafter, up until the aerosolized OVA challenge. A second series consists of groups of mice receiving different doses of W-tide given either intraperitoneally, intra-venously, subcutaneously, intramuscularly, orally, or via any other mode of administration, at the initial sensitization, and at different dosing schedules thereafter, up until the aerosolized OVA challenge. A third series of mice, serving as a positive control, consists of groups treated with either mouse IL-10 intraperitoneally, anti-IL4 antibodies intraperitoneally, or anti-IL5 antibodies intraperitoneally at the initial sensitization and at different dosing schedules thereafter, up until the aerosolized OVA challenge.

Animals are subsequently analyzed at different time points after the aerosolized OVA challenge for pulmonary function, cellular infiltrates in bronchoalveolar lavage (BAL), histological examination of lungs, and measurement of serum PA-specific IgE titers.

References

Ausubel, F. M., R. Brent, R. E. Kingston, D. D. Moore, et al. 1987. Current protocols in molecular biology. John Wiley & Sons, New York.
Bacon, K. B., R. D. Camp, F. M. Cunningham, and P. M. Woollard. 1988. Contrasting in vitro lymphocyte chemotactic activity of the hydroxyl enantiomers of 12-hydroxy-5,8,10,14-eicosatetraenoic acid. Br J Pharmacol. 95:966-74.
Bae et al. “Identification of novel chemoattractant peptides for human leukocytes,” Blood, 1 May 2001 97(9):2854-2862
Baek S H et al., “Identification of the peptides that stimulate the phosphoinositide hydrolysis in lymphocyte cell lines from peptide libraries,” J Biol Chem, 1996; 271:8170-8175
Baggiolini M. et al “Interleukin-8 and related chemotactic cytokines-CXC and CC chemokines” Adv Immunol 1994;55:97-179
Bao et al., Genomics 13: 437 (1992)
Bender, A., M. Sapp, G. Schuler, R. M. Steinman, et al. 1996. Improved methods for the generation of dendritic cells from nonproliferating progenitors in human blood. J Immunol Methods. 196:121-35.
Bokoch G M “Chemoattractant signaling and leukocyte activation” BLOOD 1995;86: 1649-1660
Campbell, J. J., E. P. Bowman, K. Murphy, K. R. Youngman, et al. 1998. 6-C-kine (SLC), a lymphocyte adhesion-triggering chemokine expressed by high endothelium, is an agonist for the MIP-3β receptor CCR7. J Cell Biol. 141:1053-9.
Carter, P. 1986. Site-directed mutagenesis. Biochem J. 237:1-7.
Cella, M., M. Salio, Y. Sakakibara, H. Langen, et al. 1999. Maturation, activation, and protection of dendritic cells induced by double-stranded RNA. J Exp Med. 189:821-9.
Chan, V. W., S. Kothakota, M. C. Rohan, L. Panganiban-Lustan, et al. 1999. Secondary lymphoid-tissue chemokine (SLC) is chemotactic for mature dendritic cells. Blood. 93:3610-6.
Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.
Dieu, M. C., B. Vanbervliet, A. Vicari, J. M. Bridon, et al. 1998. Selective recruitment of immature and mature dendritic cells by distinct chemokines expressed in different anatomic sites. J Exp Med. 188:373-86.
Drowning, D. D., Pan, Z. K., Prossnitz, E. R., Ye, R. D., 1997. Cell type- and development stage-specific activation of NF-kappaB by fMet-Leu-Phe in myeloid cells. J Biol Chem. 272:7995-8001.
Durstin et al., Biochem. Biophys. Res. Commun. 201. 174. 1994.
Forssmann, U., M. B. Delgado, M. Uguccioni, P. Loetscher, et al. 1997. CKβ8, a novel CC chemokine that predominantly acts on monocytes. FEBS Lett. 408:211-6.
Gao and M. Murphy, J Biol. Chem. 268: 25395, 1993.
Goodwin, J., RH. U.S. Pat. No. 5,284,753. 1994. Multiple-site chemotactic test apparatus and method.
Horuk, Trends Pharm. Sci., 15:159-165, 1994.
Kaufman, R. J. 1990. Vectors used for expression in mammalian cells. Methods Enzymol. 185:487-511.
Kellermann, S. A., S. Hudak, E. R. Oldham, Y. J. Liu, et al. 1999. The CC chemokine receptor-7 ligands 6Ckine and macrophage inflammatory protein-3β are potent chemoattractants for in vitro- and in vivo-derived dendritic cells. J Immunol. 162:3859-64.
Kraal, G., M. Breel, M. Janse, and G. Bruin. 1986. Langerhans' cells, veiled cells, and interdigitating cells in the mouse recognized by a monoclonal antibody. J Exp Med. 163:981-97.
Kriegler, M. 1990. Gene transfer and expression: A laboratory manual. Stockton Press, New York. 242 pp.
Le et al., “Pleiotropic roles of formyl peptide receptors,” Cytokine and Growth Factor Reviews 12:91-105 (2001)
Le, Y., Hu, J., Gong, W., Shen, W., Le, B., Dunlop, N. M., et al. Expression of functional formyl peptide receptors by human astrocytoma cell lines. 2000 J Neuroimmunol. 111:102-8.
Meikle, J. 2002. Pioneering gene treatment gives frail toddler a new lease of life. In The Guardian, London.
Murphy et al., J. Biol. Chem. 267: 7637 (1992);
Murphy, “The N-formylpeotide chemotactic receptors, Chemoattractant ligands and their receptors,” CRC Press, Boca Raton, p. 269 (1996)
Nabel and Nabel, U.S. Pat. No. 5,328,470, 1994
Nomura et al., Int Immilnol. 5: 1239 (1993)
Penfold, M. E., D. J. Dairaghi, G. M. Duke, N. Saederup, et al. 1999. Cytomegalovirus encodes a potent alpha chemokine. Proc Natl Acad Sci USA. 96:9839-44.
Prieschl E E et al. “The nomenclature of chemokines” Int Arch Allergy Immunol 1995; 107:475-483;
Prossnitz and Ye, Pharmacol Ther. 74: 73 (1997)
Romani, N., D. Reider, M. Heuer, S. Ebner, et al. 1996. Generation of mature dendritic cells from human blood. An improved method with special regard to clinical applicability. J Immunol Methods. 196:137-51.
Rossi, D., and A. Zlotnik. 2000. The Biology of Chemokines and their Receptors. Annu. Rev. Immunol. 18:217-242.
Sambrook, J. 1989. Molecular cloning: a laboratory manual. Cold Spring Harbor Laboratory, Cold Spring Harbor.
Seo, J. K., et al., 1997. J. Immunol. 158: 1895-1901.
Spatola (1983) in Chemistry and Biochemistry of Amino Acids, Peptides and Proteins, Vol. 7, pp 267-357, “Peptide Backbone Modifications,” Marcell Dekker, NY
Tice et al., U.S. Pat. No. 5,942,252, 1999
Verdijk, R. M., T. Mutis, B. Esendam, J. Kamp, et al. 1999. Polyriboinosinic polyribocytidylic acid (poly(I:C)) induces stable maturation of functionally active human dendritic cells. J Immunol. 163:57-61.
Wells, J. A., M. Vasser, and D. B. Powers. 1985. Cassette mutagenesis: an efficient method for generation of multiple mutations at defined sites. Gene. 34:315-23.
Ye et al., Biochem. Xophys. Res. Commul 7-184: 582 (1992)
Zoller, M. J., and M. Smith. 1987. Oligonucleotide-directed mutagenesis: a simple method using two oligonucleotide primers and a single-stranded DNA template. Methods Enzymol. 154:329-50.
WO 03/064447 A2
U.S. application Ser. No. 10/141,508, filed on May 7, 2002

1. A method of modulating an immune response in a subject comprising:
administering to the subject an amount sufficient to modulate an immune response in the subject of
at least one W-peptide or a conservative variant or a functional fragment thereof and
at least one antigen.

2. The method of claim 1, wherein the immune response is an antibody-mediated immune response.

3. The method of claim 1, wherein the immune response is a cell-mediated immune response.

4. The method of claim 1, wherein the W-peptide attracts a dendritic cell.

5. The method of claim 1, wherein the W-peptide and the antigen are co-administered.

6. The method of claim 1, wherein the W-peptide and the antigen are administered separately.

7. The method of claim 1, wherein the antigen comprises a polynucleotide encoding the antigen.

8. The method of claim 1, wherein the antigen is a polypeptide

9. The method of claim 1, wherein the antigen is a polypeptide from a pathogen.

10. The method of claim 9, wherein the pathogen is Hepatitis or Influenza.

11. The method of claim 1, wherein the antigen is a tumor antigen.

12. The method of claim 1, wherein the antigen is a self antigen in an auto-immune disease.

13. The method of claim 1, wherein the administering further comprises administering a conventional adjuvant.

14. The method of claim 11, wherein the conventional adjuvant is selected from the group consisting of Alum, incomplete Freund's adjuvant, CpG oligonucleotides, a bacterial capsular polysaccharide, dextran, IL-12, GM-CSF, CD40 ligand, IFN-γ, IL-1, IL-2, IL-3, IL-4, IL-10, IL-13, IL-18 and a cytokine, or fragments thereof.

15. The method of claim 1, wherein the administering further comprises administering a multivalent carrier.

16. The method of claim 15, wherein the multivalent carrier is selected from the group consisting of a bacterial capsular polysaccharide, a dextran and a polynucleotide vector.

17. The method of claim 16, wherein the bacterial capsular polysaccharide is a Pneumococci, Streptococci or Meningococci polysaccharide.

18. The method of claim 1, further comprises administering a pharmaceutical carrier.

19. The method of claim 1, wherein the administering is into a solid tumor.

20. The method of claim 1, wherein the administering is into tissue surrounding a solid tumor.

21. The method of claim 1, wherein the administering is injecting, inhaling, or oral.

22. The method of claim 1, which comprises administering the W-peptide and the antigen at least twice.

23. The method of claim 22, which comprises administering the W-peptide and antigen at the same site.

24. The method of claim 1, wherein the W-peptide comprises a polynucleotide encoding the W-peptide.

25. The method of claim 1, further comprising administering at least two W-peptides.

26. The method of claim 25, wherein the W-tides are linked.

27. The method of claim 1, wherein the W-peptide is formulated in a sustained release pharmaceutical composition.

28. The method of claim 1, wherein the W-peptide and antigen are co-administered.

29. The method of claim 1, wherein the W-peptide and the antigen are administered separately.

30. A method of producing antibodies to an antigen in a subject comprising:
administering to the subject at least one antigen and at least one W-peptide or a conservative variant or a functional fragment thereof, in an amount sufficient to elicit production of antibodies to the antigen in the subject.

31. The method of claim 30, wherein the administering increases the titer of antigen-specific antibodies in the subject by at least two fold.

32. The method of claim 30, wherein the antibody is a monoclonal antibody.

33. The method of claim 30, wherein the antigen and the W-peptide are co-administered.

34. The method of claim 30, wherein the antigen and the W-peptide are administered separately.

35. The method of claim 30, wherein the antigen is selected from the group consisting of peptide, polypeptide, chemical compound, microbial pathogen, bacteria, virus, recombinant cell, glycoproteins, lipoproteins, glycopeptides, lipopeptides, toxoids, carbohydrates, tumor-specific antigens, and other immunogenic components of pathogens

36. The method of claim 30, wherein the antigen is a polypeptide from a pathogen.

37. The method of claim 36, wherein the pathogen is Anthrax.

38. The method of claim 30, wherein the antigen is a recombinant Anthrax protective antigen.

39. The method of claim 30, wherein the pathogen is Hepatitis or Infuenza.

40. The method of claim 30, wherein the administering further comprises administering a conventional adjuvant.

41. The method of claim 40, wherein the conventional adjuvant is selected from the group consisting of Alum, incomplete Freund's adjuvant, CpG oligonucleotides, a bacterial capsular polysaccharide, dextran, IL-12, GM-CSF, CD40 ligand, IFN-γ, IL-1, IL-2, IL-3, IL-4, IL-10, IL-13, IL-18 and a cytokine, or fragments thereof.

42. The method of claim 30, comprising administering at least twice.

43. The method of claim 42, wherein the administrations are at the same site.

44. The method of claim 30, wherein the W-peptide is a polypeptide-Ig fusion polypeptide.

45. The method of claim 30, wherein the W-peptide and the antigen are fused together.

46. The method of claim 30, wherein the W-peptide and the antigen are chemically cross-linked.

47. A composition comprising:
at least one W-peptide or conservative variant or a functional fragment thereof;
at least one antigen; and
a pharmaceutically acceptable carrier.

48. The composition of claim 47, further comprising a conventional adjuvant.

49. The composition of claim 48, wherein the conventional adjuvant is selected from the group consisting of Alum, incomplete Freund's adjuvant, CpG oligonucleotides, a bacterial capsular polysaccharide, dextran, IL-12, GM-CSF, CD40 ligand, IFN-γ, IL-1, IL-2, IL-3, IL-4, IL-10, IL-13, IL-18 and a cytokine, or fragments thereof.

50. The composition of claim 47, wherein the pharmaceutically acceptable carrier is selected from the group consisting of mannitol, lactose, and magnesium stearate.

51. The composition of claim 47, wherein the pharmaceutically acceptable carrier is a multivalent carrier.

52. The composition of claim 51, wherein the multivalent carrier is selected from the group consisting of a bacterial capsular polysaccharide, dextran, and a genetically engineered vector.

53. The composition of claim 52, wherein the bacterial capsular polysaccharide is a Pneumococci, Streptococci or Meningococci polysaccharide.

54. The composition of claim 53, wherein the multivalent carrier is linked to the W-peptide, the antigen and a conventional adjuvant.


SEQUENCE LISTING (28 sequences)

SEQ ID NO: 1 - protein, 6 aa, Artificial Sequence
  W-tide
HFYLPM

SEQ ID NO: 2 - protein, 6 aa, Artificial Sequence
  W-tide
MFYLPM

SEQ ID NO: 3 - protein, 6 aa, Artificial Sequence
  W-tide
HFYLPM

SEQ ID NO: 4 - protein, 6 aa, Artificial Sequence
  W-tide
WKYMVM

SEQ ID NO: 5 - protein, 6 aa, Artificial Sequence
  W-tide
WKGMVM

SEQ ID NO: 6 - protein, 6 aa, Artificial Sequence
  W-tide
WKYMGM

SEQ ID NO: 7 - protein, 6 aa, Artificial Sequence
  W-tide
WKYMVG

SEQ ID NO: 8 - protein, 6 aa, Artificial Sequence
  W-tide
WRYMVM

SEQ ID NO: 9 - protein, 6 aa, Artificial Sequence
  W-tide
WEYMVM

SEQ ID NO: 10 - protein, 6 aa, Artificial Sequence
  W-tide
WHYMVM

SEQ ID NO: 11 - protein, 6 aa, Artificial Sequence
  W-tide
WDYMVM

SEQ ID NO: 12 - protein, 6 aa, Artificial Sequence
  W-tide
WKHMVM

SEQ ID NO: 13 - protein, 6 aa, Artificial Sequence
  W-tide
WKEMVM

SEQ ID NO: 14 - protein, 6 aa, Artificial Sequence
  W-tide
WKWMVM

SEQ ID NO: 15 - protein, 6 aa, Artificial Sequence
  W-tide
WKRMVM

SEQ ID NO: 16 - protein, 6 aa, Artificial Sequence
  W-tide
WKDMVM

SEQ ID NO: 17 - protein, 6 aa, Artificial Sequence
  W-tide
WKFMVM

SEQ ID NO: 18 - protein, 6 aa, Artificial Sequence
  W-tide
WKYMYM

SEQ ID NO: 19 - protein, 6 aa, Artificial Sequence
  W-tide
WKYMXM

SEQ ID NO: 20 - protein, 6 aa, Artificial Sequence
  W-tide
WKYMVE

SEQ ID NO: 21 - protein, 6 aa, Artificial Sequence
  W-tide
WKYMVV

SEQ ID NO: 22 - protein, 6 aa, Artificial Sequence
  W-tide
WKYMVR

SEQ ID NO: 23 - protein, 6 aa, Artificial Sequence
  W-tide
WKYMVW

SEQ ID NO: 24 - protein, 5 aa, Artificial Sequence
  W-tide
WKYMV

SEQ ID NO: 25 - protein, 5 aa, Artificial Sequence
  W-tide
KYMVM

SEQ ID NO: 26 - protein, 4 aa, Artificial Sequence
  W-tide
KYMV

SEQ ID NO: 27 - protein, 4 aa, Artificial Sequence
  W-tide
YMVM

SEQ ID NO: 28 - protein, 3 aa, Artificial Sequence
  W-tide
MVM